(19) United States
(12) Patent Application Publication
(10) Pub. No.: US 2025/0204810 A1
(43) Pub. Date: Jun. 26, 2025

(54) APPARATUS FOR ESTIMATING HUMAN POSTURE AND METHOD THEREOF

(71) Applicant: Research & Business Foundation Sungkyunkwan University, Suwon-si (KR)

(72) Inventors: Jangwon LEE, Seoul (KR); Jong Min LEE, Seoul (KR); In Pyo SONG, Seoul (KR)

(73) Assignee: Research & Business Foundation Sungkyunkwan University, Suwon-si (KR)

(21) Appl. No.: 18/979,379

(22) Filed: Dec. 12, 2024

(30) Foreign Application Priority Data
Dec. 20, 2023 (KR) 10-2023-0186741
Nov. 26, 2024 (KR) 10-2024-0170269

(51) Int. Cl. A61B 5/11; A61B 5/00

(52) U.S. Cl. CPC A61B 5/1116; A61B 5/0077; A61B 5/1128; A61B 5/7267; A61B 5/7278; A61B 2576/00

(57) ABSTRACT

The present invention relates to an apparatus and a method for estimating human posture. The method for estimating human posture comprises generating a plurality of motion-aware heatmaps for each joint based on a plurality of previously input images corresponding to continuous time, generating intersection heatmaps by considering motions between motion-aware heatmaps at different time points from among the plurality of motion-aware heatmaps and estimating a human posture based on the intersection heatmap.

### CROSS-REFERENCE TO RELATED APPLICATIONS

This application claims the priority benefit of Korean Patent Application No. 10-2023-0186741 filed on Dec. 20, 2023, in the Korean Intellectual Property Office and Korean Patent Application No. 10-2024-0170269 filed on Nov. 26, 2024, in the Korean Intellectual Property Office, the disclosures of which are incorporated herein by reference.

### BACKGROUND

### 1. Field

The present invention relates to an apparatus for estimating human posture and a method thereof.

### 2. Description of the Related Art

The conventional technology for estimating human posture is based on a 2D camera, and has drawn much attention because it can estimate human posture using relatively inexpensive equipment, but the technology for estimating human posture based on a 2D camera is designed based on a still image, and thus has a problem in that it is vulnerable to dynamic movement, and it is difficult to accurately estimate human posture when the human posture changes or moves.

In addition, there is a problem in that a jittering occurs in the process of estimating the positions of posture feature points in an image acquired through a 2D camera.

Here, the jittering is caused by image noise, a change in lighting, or a fine movement of a camera, and research on a technology for stabilizing the jittering is insufficient.

Therefore, there is a need for technologies that improve the accuracy and stability of the human posture estimation technology using a 2D camera.

### SUMMARY

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter.

An object of the present invention is to provide an apparatus for estimating human posture and a method thereof, which estimate human posture on the basis of motion-aware heatmaps generated from continuous images.

To solve the above-described problems, an apparatus for estimating human posture and a method thereof are provided.

A method for estimating human posture comprises generating a plurality of motion-aware heatmaps for each joint based on a plurality of previously input images corresponding to continuous time, generating intersection heatmaps by considering motions between motion-aware heatmaps at different time points from among the plurality of motion-aware heatmaps and estimating a human posture based on the intersection heatmap.

The generating of the intersection heatmaps comprises generating the intersection heatmaps based on motion-aware heatmaps at each of a current time point, a past time point, and a future time point, which are different time points

The generating of the plurality of motion-aware heatmaps comprises generating a motion-aware heatmap at the current time point, a past motion-aware heatmap at a past time point relative to the current time point, and a future motion-aware heatmap at a future time point relative to the current time point.

The generating of the plurality of motion-aware heatmaps comprises when there are a plurality of motion-aware heatmaps of each of the past time point and the future time point, generating current motion-aware heatmaps with respect to the current time point, a first past motion-aware heatmap at a first past time point relative to the current time point, a second past motion-aware heatmap at a second past time point earlier than the first past time point, a first future motion-aware heatmap at a first future time point relative to the current time point, and a second future motion-aware heatmap at a second future time point later than the first future time point.

The generating the intersection heatmaps comprises generating a first intersection heatmap by averaging a product between the second past motion-aware heatmaps and the first past motion-aware heatmap and a product between the first past motion-aware heatmap and the current motion-aware heatmaps and generating a second intersection heatmap by averaging a product between the second future motion-aware heatmap and the first future motion-aware heatmap and a product between the first future motion-aware heatmap and current the motion-aware heatmap.

The method for estimating human posture further comprises calculating weights for each of the plurality of motion-aware heatmaps, wherein the generating the intersection heatmap comprises generating the intersection heatmaps by reflecting the weights of each of the plurality of motion-aware heatmaps.

The estimating the human posture comprises generating a combined heatmap by combining the intersection heatmaps and estimating the human posture on the basis of the combined heatmap.

The estimating the human posture on the basis of the combined heatmap comprises generating a merged heatmap by merging the plurality of motion-aware heatmaps, extracting an offset and a mask based on the combined heatmap and estimating the human posture based on the merged heatmap, the offset, and the mask.

The generating the plurality of motion-aware heatmaps comprises extracting a motion vector of a joint keypoint from each of the plurality of images and generating the plurality of motion-aware heatmaps based on a magnitude and a direction of the motion vector of the joint keypoint.

The generating the plurality of motion-aware heatmaps comprises learning to generate the motion-aware heatmaps by using regression loss.

An apparatus for estimating human posture comprises a processor, wherein the processor comprises a generator configured to generate a plurality of motion-aware heatmaps for each joint based on a plurality of previously input images corresponding to continuous time, and to generate intersection heatmaps by considering motions between motion-aware heatmaps at different time points from among the plurality of motion-aware heatmaps and an estimator configured to estimate human posture in an image at the current time point based on the intersection heatmap.

The generator is further configured to generate the intersection heatmaps based on motion-aware heatmaps at each of a current time point, a past time point, and a future time point, which are different time points.

The generator is further configured to generate a motion-aware heatmap at the current time point, a past motion-aware heatmap at a past time point relative to the current time point, and a future motion-aware heatmap at a future time point relative to the current time point. [0023] the generator is further configured to, when there are a plurality of motion-aware heatmaps of each of the past time point and the future time point, generate current motion-aware heatmaps with respect to the current time point, a first past motion-aware heatmap at a first past time point relative to the current time point, a second past motion-aware heatmap at a second past time point earlier than the first past time point, a first future motion-aware heatmap at a first future time point relative to the current time point, and a second future motion-aware heatmap at a second future time point later than the first future time point.

The generator is further configured to generate a first intersection heatmap by averaging a product between the second past motion-aware heatmaps and the first past motion-aware heatmap and a product between the first past motion-aware heatmap and the current motion-aware heatmaps and generate a second intersection heatmap by averaging a product between the second future motion-aware heatmap and the first future motion-aware heatmap and a product between the first future motion-aware heatmap and current the motion-aware heatmap.

The generator is further configured to calculate weights for each of the plurality of motion-aware heatmaps, and generate the intersection heatmaps by reflecting the weights of each of the plurality of motion-aware heatmaps.

The estimator is further configured to generate a combined heatmap by combining the intersection heatmaps and estimate the human posture on the basis of the combined heatmap.

The estimator is further configured to generate a merged heatmap by merging the plurality of motion-aware heatmaps, extract an offset and a mask based on the combined heatmap and estimate the human posture based on the merged heatmap, the offset, and the mask.

The generator is further configured to extract a motion vector of a joint keypoint from each of the plurality of images and generate the plurality of motion-aware heatmaps based on a magnitude and a direction of the motion vector of the joint keypoint.

The processor further comprises a learner configured to learn to generate the motion-aware heatmaps using regression loss.

According to the above-described apparatus for estimating human posture and method thereof, posture of a human motion may be estimated by generating intersection heatmaps based on a current motion-aware heatmaps at a current time, a past motion-aware heatmaps at a past time, and a future motion-aware heatmaps at a future time.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a block diagram illustrating the configuration of an apparatus for estimating human posture according to an embodiment of the present disclosure.

FIG. 2 is a diagram illustrating the process of estimating human posture according to an embodiment of the present disclosure.

FIGS. 3A and 3B are diagrams illustrating the generation of motion-aware heatmaps according to an embodiment of the present disclosure.

FIG. 4 is a diagram illustrating motion-aware heatmaps according to a regression loss according to an embodiment of the present disclosure.

FIG. 5 is a diagram illustrating estimation of human posture according to an embodiment of the present disclosure

FIG. 6 is a flowchart illustrating a method for estimating human posture according to an embodiment of the present disclosure.

FIG. 7 is a flowchart illustrating generation of motion-aware heatmaps according to an embodiment of the present disclosure.

FIG. 8 is a flowchart illustrating generation of intersection heatmaps according to an embodiment of the present disclosure.

FIG. 9 is a flowchart illustrating estimation of human posture according to an embodiment of the present disclosure.

Throughout the drawings and the detailed description, the same reference numerals may refer to the same, or like, elements. The drawings may not be to scale, and the relative size, proportions, and depiction of elements in the drawings may be exaggerated for clarity, illustration, and convenience.

### DETAILED DESCRIPTION

The advantages and features of the present invention, and the methods for achieving them, will become apparent by referring to the embodiments described below in conjunction with the accompanying drawings. However, the present invention is not limited to the embodiments disclosed herein and may be implemented in various other forms. The embodiments are merely provided to ensure the completeness of the disclosure and to fully convey the scope of the invention to those skilled in the art to which the present invention pertains. The present invention shall be defined only by the scope of the claims.

The terms used in this specification will be briefly explained, followed by a detailed description of the present invention.

The terms used in the present invention have been selected, to the extent possible, from general terms widely used at present while considering their functionality within the invention. However, these terms may vary depending on the intention of those skilled in the art, judicial precedents, or the emergence of new technologies. In certain cases, terms may have been arbitrarily defined by the applicant. In such cases, the meaning of the terms will be clearly described in the relevant portions of the invention. Therefore, the terms used in the present invention should not be interpreted as simple labels but should be defined based on their meanings and the overall context of the invention.

Throughout the specification, when a certain portion is described as “including” a particular component, unless specifically stated otherwise, it implies that additional components may be included and does not exclude other components. Furthermore, the terms such as “part,” “module,” and “unit” used in this specification refer to units that process at least one function or operation. These units may be implemented as hardware components such as software, FPGAs, or ASICs, or as combinations of software and hardware. However, the terms “part,” “module,” and “unit” are not limited to software or hardware alone. These terms may also refer to components stored in addressable storage media and configured to execute on one or more processors. For example, terms such as “part,” “module,” and “unit” may encompass software components, object-oriented software components, class components, task components, processes, functions, properties, procedures, subroutines, program code segments, drivers, firmware, microcode, circuits, data, databases, data structures, tables, arrays, and variables.

The following provides a detailed description of embodiments of the present invention with reference to the accompanying drawings so that those skilled in the art to which the present invention pertains can readily implement the invention. Parts irrelevant to the explanation of the invention have been omitted from the drawings for clarity.

Terms such as “first,” “second,” and the like, which include ordinals, may be used to describe various components, but these components are not limited by such terms. These terms are used solely to distinguish one component from another. For example, without departing from the scope of the present invention, a “first” component may be referred to as a “second” component, and similarly, a “second” component may be referred to as a “first” component. Additionally, the term “and/or” includes both combinations of multiple associated items and any one of the multiple associated items.

The apparatus for estimating human posture according to the present invention may estimate human posture on the basis of a motion-aware heatmaps generated from continuous images.

Although the apparatus and method for estimating human posture according to the present disclosure described below have been described based on a two-dimensional (2D) image for convenience, it should be understood that the apparatus and method may be applied to a three-dimensional (3D) image. In the specification of the present invention, since the content described based on a two-dimensional image may be interpreted as a description based on a three-dimensional image without any particular issue, it is clear that the human posture estimation technology of the present invention may be implemented in three dimensions as well. For example, in a device, a two-dimensional vector with x and y axes can be interpreted as a three-dimensional vector with x, y, and z axes. The information derived from each vector encompasses not only direction and magnitude but also rotational information in three-dimensional space. Likewise, a 2D heatmap on a plane can be naturally replaced and understood as a 3D heatmap, which further includes depth and spatial location information of an object.

In addition, the apparatus and method for estimating human posture according to the present disclosure described below are, for convenience, explained as being based on data collected from an external observer's viewpoint. However, it should be noted that they can also be implemented based on data collected from an egocentric view. Specifically, the apparatus for estimating human posture of the present disclosure may also be applied using image data captured from the user's own perspective when the user wears a device such as a virtual reality (VR) or augmented reality (AR) apparatus.

In addition, the apparatus and method for estimating human posture according to the present disclosure described below may be seamlessly integrated into and applied across various domains. For instance, the apparatus and method for estimating human posture according to an exemplary embodiment of the present invention can be utilized in diverse computer vision domains such as object detection and tracking. When phenomena like rapid motion changes, blur, or occlusion occur, a heatmap incorporating the motion information of an object may be used to enable more precise and sophisticated analysis.

Hereinafter, an apparatus for estimating human posture according to an embodiment of the present invention will be described with reference to the drawings.

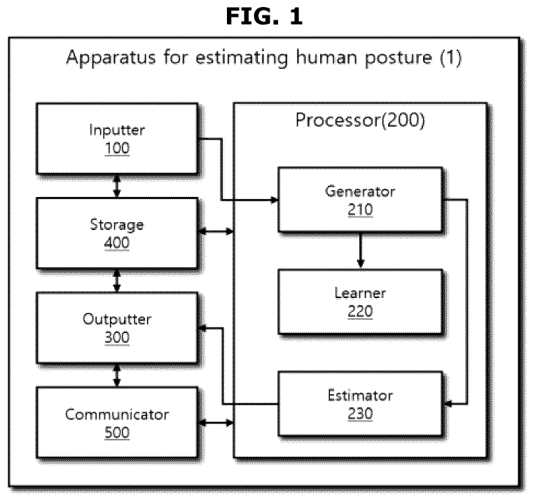

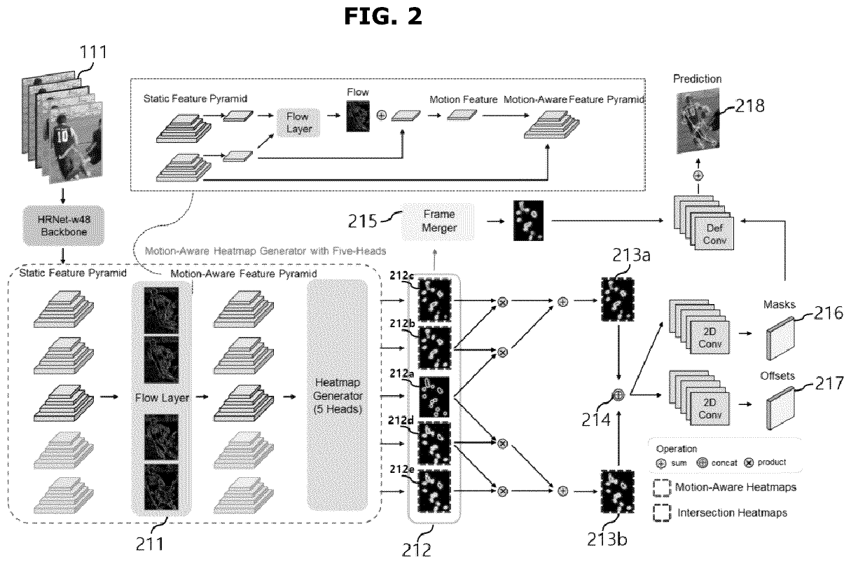

FIG. 1 is a block diagram for describing a configuration of an apparatus for estimating human posture according to an embodiment of the present disclosure, and FIG. 2 is a diagram for describing a process of estimating human posture according to an embodiment of the present disclosure.

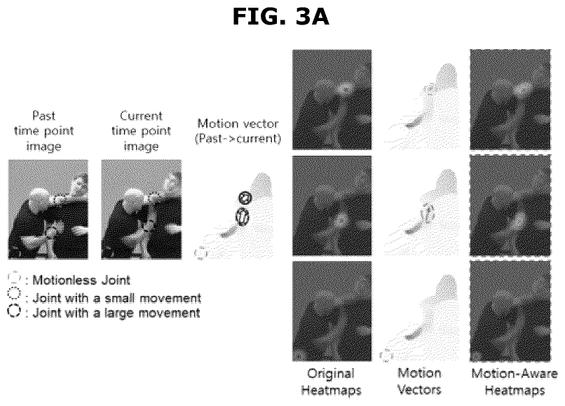

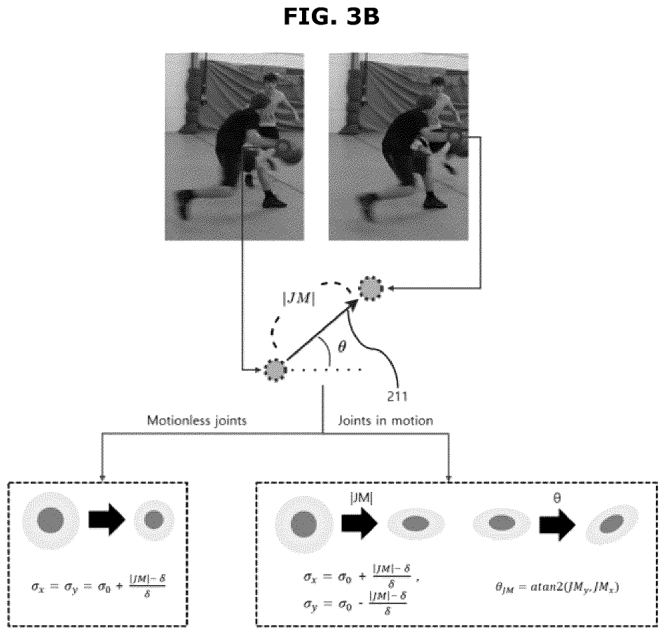

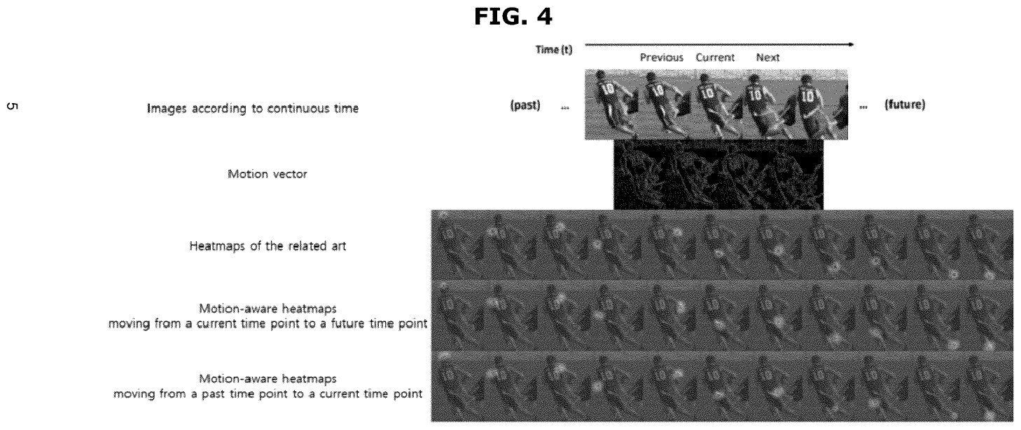

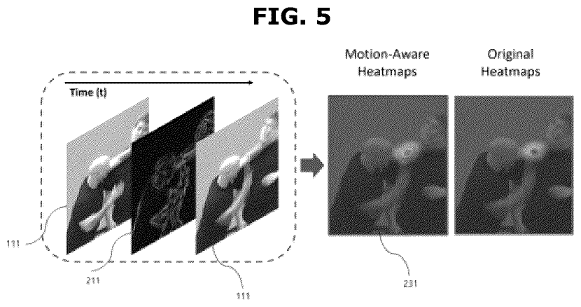

FIGS. 3A and 3B are diagrams for describing generation of motion-aware heatmaps according to an embodiment of the present disclosure, FIG. 4 is a diagram for describing motion-aware heatmaps according to a regression loss according to an embodiment of the present disclosure, and FIG. 5 is a diagram for describing estimation of human posture according to an embodiment of the present disclosure.

As shown in FIG. 1, an apparatus for estimating human posture 1 according to an embodiment of the present invention includes an inputter (e.g., including input circuitry) 100, a processor 200, an outputter (e.g., including output circuitry) 300, a storage 400, and a communicator 500.

The communicator 500 may allow the inputter 100, the processor 200, the outputter 300, and the storage 400 to transmit and receive data to and from each other.

The communicator 500 supports both wired and wireless communication networks. For example, the communicator 500 may utilize or integrate a wired/wireless Internet network. The wired network includes Internet networks such as cable networks or public telephone networks (PSTN), while the wireless communication network encompasses technologies such as CDMA, WCDMA, GSM, evolved packet core (EPC), long term evolution (LTE), Wibro, and 5G communication networks, among others. Naturally, the communication network 130, as described in an embodiment of the present disclosure, is not limited to these examples. It may also function as an access network for next-generation mobile communication systems, including cloud computing networks under a cloud computing environment, 5G networks, and similar systems. For instance, if the communicator 500 operates as a wired communication network, its access point may connect to an exchange station of a telephone station. Conversely, if it functions as a wireless communication network, the access point may connect to an SGSN or a Gateway GPRS Support Node (GGSN) managed by a communication provider to process data. It may also connect to various repeaters such as Base Station Transmission (BST), NodeB, or e-NodeB to manage data transmission.

The inputter 100 receives a plurality of images 111 according to continuous time from a user.

The plurality of images 111 are continuous frames, and the images and the frames may be used interchangeably.

In an embodiment of the present invention, it is described that five images are used, but the present invention is not limited thereto.

The processor 200 may include a generator 210, a learner 220, and an estimator 230.

As shown in FIG. 2, the generator 210 may extract a motion vector 211 of a joint keypoint from each of the plurality of images.

Specifically, the generator 210 may extract intermediate features for each of the plurality of images using a backbone network.

Here, the backbone network may be a HRNet-w48.

The generator 210 may extract a motion vector 211 for the joint keypoint based on the extracted intermediate feature.

If the intermediate feature layer of each image is denoted as F^{h}ᵢ (where i represents an image at the current time point and h indicates the hierarchical order of the feature pyramid), ‘c’ represents the image at the current time point, and ‘a’ represents an adjacent image moving relative to the image at the current time point, and if each static feature pyramid consists of four feature layers F¹, F², F³ᵢ, and F⁴ ranging from high resolution to low resolution, then the generator 210 may take continuous images as inputs and extract a motion feature (F¹_{c→a}) using the following Equation 1.

$${F}_{c \to a}^{h} = ( {F}_{c}^{h} , {F}_{a}^{h} ) \qquad [ Equation 1 ]$$

Here, F^{h}_{c→a} denotes a motion feature moving from an image ‘c’ at the current time to an adjacent image ‘a’, F^{h}_{c} denotes a feature layer of the image at the current time, F^{h}ₐ denotes a feature layer of the adjacent image to the image at the current time, and ‘M’ denotes a representation flow for action recognition for extracting a motion vector from an intermediate feature.

In order to extract the motion feature F¹_{c→a}, the generator 210 may use a feature of a high resolution, and features F²ₐ, F³ₐ, and F⁴ₐ of other low resolutions may be maintained in adjacent images.

Here, the generator 210 may extract the motion vector 211 using a flow layer. According to an embodiment, the generator 210 may use an optical flow-based model, and the generator 210 may capture dynamic characteristics more accurately by using an optical flow-based model capable of providing a magnitude and a direction of the motion vector 211 of each joint.

The generator 210 may generate a plurality of motion-aware heatmaps 212 at different time points based on the respective motion vectors 211 extracted from the plurality of images 111.

The generator 210 may generate a plurality of motion-aware heatmaps 212 based on the magnitude and direction of the motion vector of the joint keypoint.

Specifically, the heatmap generator 210 may generate a motion recognition pyramid feature to have a hierarchical structure for a feature map.

After extracting the motion feature, the generator 210 may replace F¹ₐ with F¹_{c→a} to generate a motion recognition pyramid feature.

The generator 210 may generate the motion-aware heatmaps 212 based on the image movement when the image ‘c’ moves from the image to the image ‘a’.

As shown in FIG. 3A, the plurality of motion-aware heatmaps 212 may be generated based on the magnitude and direction of the motion vector at each joint keypoint using the extracted motion vector 211, unlike the original heatmaps produced by conventional technology.

Here, the generator 210 may individually adjust the standard deviation according to the magnitude of the motion vector for each axis, rotate the Gaussian kernel according to the direction of the joint motion vector, and arrange the adjusted Gaussian kernel on all joint keypoints of the image.

A process of generating the motion-aware heatmaps 212 according to an embodiment will be described with reference to FIG. 3B. According to an embodiment, the generator 210 may generate the motion-aware heatmaps 212 based on a Gaussian heatmap.

The generator 210 may generate motion-aware heatmaps 212 for each joint by using the magnitude |JM| and the direction θ of the motion vector 211 extracted for each of several joints.

In the case of joints with little or no movement (motionless joints), the joint may have a motion vector 211 having a size equal to or less than a specific threshold value. When the magnitude of the motion vector 211 is less than or equal to a preset value, the Gaussian heatmap may be represented in a small circular shape. That is, when the size of the motion vector 211 extracted from one joint is less than or equal to a preset threshold, the generator 210 may generate the motion-aware heatmaps 212 in the form of a small circular Gaussian heatmap.

In the case of joints in motion, the joint may have a motion vector 211 having a magnitude greater than a specific threshold value. When the size of the motion vector 211 exceeds a preset value, the Gaussian heatmap may appear in the form of an ellipse in which the x-axis and the y-axis have different sizes. That is, when the size of the motion vector 211 extracted from one joint exceeds a preset threshold, the generator 210 may generate the motion-aware heatmaps 212 in the form of an elliptical Gaussian heatmap in which the x-axis and y-axis sizes are different from each other. Accordingly, in the case of a joints in motion, the generator 210 may generate the motion-aware heatmaps 212 that is stretched to a specific size or rotated at a specific angle to reflect the movement direction and strength of the joint.

The generator 210 may generate heatmaps reflecting the movement of the joint differently for each joint through the above-described method. In addition, the heatmaps of each joint generated in this manner may be used in a learning process to be described later.

The generator 210 may generate the motion-aware heatmaps 212 from each motion vector 211 using a plurality of heads, which are heatmap generators, in order to simultaneously generate the motion-aware heatmaps 212.

The generator 210 may generate motion-aware heatmaps 212 at each of a current time point, a past time point, and a future time point, which are different time points.

The generator 210 may generate motion-aware heatmaps at a current time point, a past motion-aware heatmaps at a past time point based on the current time point, and a future motion-aware heatmaps at a future time point based on the current time point.

More specifically, as shown in FIG. 3A, the motion-aware heatmaps 212 may include a motion-aware heatmap (H_{c}) 212a at the current time point, a first past motion-aware heatmap H_{c→p1} 212b at a first past time point relative to the current time point, a second past motion-aware heatmap H_{c→p2} 212c at a second past time point earlier than the first past time point, a first future motion-aware heatmap H_{c→n1} 212d at a first future time point relative to the current time point and a second future motion-aware heatmap H_{c→n2} 212e at a second future time point later than the first future time point.

The motion-aware heatmaps ‘H’ is a heatmap for j joints, and the motion-aware heatmaps of each joint may be represented as Hᵢ.

In an embodiment of the present disclosure, it is described that the motion vector 211 and the motion-aware heatmaps 212 are generated by receiving the plurality of images 111, but the motion-aware heatmaps 212 may be generated by inputting the motion vector 211 and the RGB image that are extracted in advance.

The generator 210 generates intersection heatmaps 213a and 213b in consideration of motions between motion-aware heatmaps of different viewpoints from among the plurality of motion-aware heatmaps 212.

The generator 210 may generate the intersection heatmaps based on the motion-aware heatmaps at each of a current time point, a past time point, and a future time point, which are different time points.

More specifically, since the degree of motion is different for each joint within each image, the generator 210 may apply a spatio-temporal weight considering the joint-specific weight of each image as a learnable parameter (αₚ₂, αₚ₁, αₙ₁, αₙ₂(α∈Rⁱ(j-dimension))).

In order to use the learned a as a weight value, the generator 210 may calculate the weight w by applying a sigmoid function that makes a value between 0 and 1 as shown in Equation 2 below.

$$σ ( x ) = \frac{1}{1 + {e}^{- x}} , w = σ ( α ) \qquad [ Equation 2 ]$$

Here, σ(x) is a sigmoid function, and w represents a weight.

The generator 210 may calculate a weight for each of the plurality of motion-aware heatmaps 212.

The generator 210 may generate the intersection heatmaps 213a and 213b by applying a weight w for each of the plurality of motion-aware heatmaps to each of the motion-aware heatmaps 212a, . . . , 212e.

The generator 210 may generate the first intersection heatmap 213a based on the second past motion-aware heatmap 212c, the first past motion-aware heatmap 212b, the first past motion-aware heatmap 212b, and the motion-aware heatmap 212a at the current time as shown in Equation 3 below.

$${H}_{inter 1 , j} = \frac{1}{2} ( {w}_{p 2 , j} \times {H}_{c \to p 2 , j} \times {w}_{p 1 , j} \times {H}_{c \to p 1 , j} + {w}_{p 1 , j} \times {H}_{c \to p 1 , j} \times {H}_{c , j} ) \qquad [ Equation 3 ]$$

Here, H_{inter1,j} is the first intersection heatmap, w_{p2,j} is a weight for the second past time point, H_{c→p2,j} is the second past motion-aware heatmap, w_{p1,j} is a weight for the first past time point, H_{c→p1,j}, is the first past motion-aware heatmap, and H_{c,j} is the motion-aware heatmap at the current time point.

In other words, the generator 210 may generate the first intersection heatmap 213a by averaging a product between the second past motion-aware heatmap 212c and the first past motion-aware heatmap 212b and a product between the first past motion-aware heatmap 212b and the current motion-aware heatmap 212a, while reflecting the weights of each of the plurality of motion-aware heatmaps.

The generator 210 may generate a second intersection heatmap 213b based on the second future motion-aware heatmap 212e, the first future motion-aware heatmap 212d, the first future motion-aware heatmap 212d, and the motion-aware heatmap 212a of the current time as shown in Equation 4 below.

$${H}_{inter 2 , j} = \frac{1}{2} ( {w}_{n 2 , j} \times {H}_{c \to n 2 , j} \times {w}_{n 1 , j} \times {H}_{c \to n 1 , j} + {w}_{n 1 , j} \times {H}_{c \to n 1 , j} \times {H}_{c , j} ) \qquad [ Equation 4 ]$$

Here, H_{inter2,j} is the second intersection heatmap, W_{n2,j} is a weight for the second future time, H_{c→n2,j} is the second future motion-aware heatmap, W_{n1,j} is a weight for the first future time, H_{c→n1,j} is the first future motion-aware heatmap, and H_{c,i} represents the motion-aware heatmap at the current time.

In other words, the generator 210 may generate the second intersection heatmap 213b by averaging a product between the second future motion-aware heatmap 212e and the first future motion-aware heatmap 212d and a product between the first future motion-aware heatmap 212d and the motion-aware heatmap 212a at the current while reflecting the weights of each of the plurality of motion-aware heatmaps.

The generator 210 may generate a combined heatmap 214 by concatenating the first intersection heatmap 213a and the second intersection heatmap 213b in which the motion information is reflected.

The generator 210 may merge the plurality of motion-aware heatmaps to generate a merged heatmap 215.

The learner 220 may be trained to generate motion-aware heatmaps using the regression loss.

The regression loss is learned based on the mean squared error (MSE) between the motion-aware heatmaps. The learner 220 may measure the difference between the motion-aware heatmaps corresponding to the probability of a joint's presence and use, as the regression loss, the extent to which the predicted motion-aware heatmaps deviate from the correct motion-aware heatmaps at the current time point.

Here, the predicted motion-aware heatmap is obtained by estimating human posture output by inputting a merged heatmap, an offset, and a mask into a deformable convolution, and represents the Prediction 218 of FIG. 2.

The learner 220 may represent an MSE loss between the predicted motion-aware heatmaps H_{pred} and the correct answer motion-aware heatmaps H_{GT} of the current viewpoint image c as a first loss value as shown in Equation 5 below.

$${Loss}_{1} = MSE ( {H}_{pred} , {H}_{GT} ) \qquad [ Equation 5 ]$$

Here, Loss1 is the first loss value, MSE is Meas Squared Error, H_{pred} is the predicted motion-aware heatmaps, and H_{GT} represents the correct answer motion-aware heatmaps at the current time.

The learner 220 may calculate Temporal weight MSE Loss between the motion-aware heatmaps H_{c→p2}, H_{c→p1}, H_{c→n1}, and H_{c→n2} having the motion information and the correct answer motion-aware heatmaps H_{GT} at the current time as a second loss value as shown in Equation 6 below.

$${Loss}_{2} = {\sum }_{k ∈ ( p 2 , p 1 , n 1 , n 2 )} \frac{1}{\vert c - k \vert} * MSE ( {H}_{c \to k} , {H}_{GT} ) \qquad [ Equation 6 ]$$

Here, Loss2 denotes the second loss value, ‘c’ denotes an image at a current time point, ‘k’ denotes an image at another time point adjacent to the image at the current time point, H_{c→k} denotes motion-aware heatmaps moving from the image at the current time point to an image at another adjacent time point, and H_{GT} denotes a correct answer motion-aware heatmaps at the current time point.

The learner 220 may represent the final loss by using the first loss value and the second loss value as shown in Equation 7 below.

$$Loss = {Loss}_{1} + {Loss}_{2} \qquad [ Equation 7 ]$$

Here, Loss denotes the final loss, Loss₁ denotes the first loss value, and Loss₂ denotes the second loss value.

As shown in FIG. 4, the apparatus for estimating human posture 1 according to an embodiment of the present disclosure may represent a signal related to joint motion in motion-aware heatmaps, unlike conventional technology.

In FIG. 4, a first row represents a plurality of images according to continuous time, a second row represents a motion vector extracted using a flow layer, a third row represents heatmaps of the related art for each joint keypoint, and a fourth row and a fifth row represent motion-aware heatmaps moving from a current time point to a future time point and motion-aware heatmaps moving from a past time point to a current time point, which are generated based on a motion vector extracted using images of past and future time points adjacent to an image of the current time point.

In this way, the learner 220 may learn to generate motion-aware heatmaps using the regression loss.

The estimator 230 may extract an offset 217 and a mask 216 based on the combined heatmap 214.

The estimator 230 may extract an offset 217 through offset convolution using the combined heatmap 214.

The estimator 230 may extract the mask 216 through mask convolution using the combined heatmap 214.

Here, each of the offset convolution and the mask convolution is composed of a plurality of 2D convolutions corresponding to the number of motion-aware heatmaps.

The estimator 230 estimates human posture based on the merged heatmap 215, the offset 217, and the mask 216.

The estimator 230 may estimate human posture in operation 231 by inputting the merged heatmap 215, the offset 217, and the mask 216 to a deformable convolution having a plurality of layers corresponding to the number of motion-aware heatmaps.

Here, although the deformable convolution is described as including a ratio of dilation of d={3, 6, 9, 12, and 15}, the embodiment is not limited thereto.

The estimator 230 may estimate the human posture by using Equation 8 below.

$${H}_{pred} = \frac{1}{5} {\sum }_{d ∈ ( 3 , 6 , 9 , 12 , 15 )}^{5} PCM ( {H}_{m} , {𝕆}_{d} , {𝕄}_{d} ) \qquad [ Equation 8 ]$$

Here, H_{pred} represents the estimated human posture, d is the dialing ratio at the deformable convolution, PCM is the pose correction module, Hₘ is the merged heatmap, O_{d} is the offset of each dilation, and M_{d} represents the mask of each dilation.

The estimator 230 may obtain a prediction of human posture as a weighted sum from an output of a final pose correction module (PCM).

Here, the pose correction module is a module that performs a series of processes of processing and adding the deformable convolution (Def conv) with respect to the dilation 3, 6, 9, 12, and 15, and in FIG. 2, the deformable convolution (Def conv) 5 is stack to represent a process of calculating the human posture estimation 218 through a+operation.

That is, the estimator 230 may estimate a human posture including the location of the human joint.

As shown in FIG. 5, the generator 220 may generate motion-aware heatmaps using the image 111 and the motion vector 211 according to continuous time.

The estimator 230 may estimate human posture using motion-aware heatmaps based on the motion of each joint keypoint, in operation 231, unlike the original heatmaps.

In this way, the apparatus for estimating human posture 1 according to the embodiment of the present invention may robustly estimate human posture and alleviate jittering.

The outputter 300 may output the estimated human posture.

The outputter 300 may include, for example, a display, a printer device, an image output terminal, a data input/output terminal, or a communication module, but is not limited thereto.

If necessary, the outputter 300 may be provided integrally with the inputter 100.

The storage 400 may store a plurality of images according to the received continuous time.

The storage 400 may store a motion vector extracted from a plurality of images and a plurality of motion-aware heatmaps generated based on the motion vector.

The storage 400 may store a plurality of intersection heatmaps generated based on the plurality of motion-aware heatmaps, and may store a mask and an offset extracted based on the plurality of intersection heatmaps.

In addition, the storage 400 may store a merged heatmap generated by merging a plurality of motion-aware heatmaps.

The storage 400 may store a heatmap for the estimated human posture.

The program stored in the storage 400 may be directly written or modified by a designer such as a programmer and then stored in the storage 400, may be received from another physical recording medium (an external memory device, a compact disk (CD), or the like) and then stored, and/or may be obtained or updated through an electronic software distribution network accessible through a wired/wireless communication network.

The storage 400 may include at least one of a main memory and an auxiliary memory. The main memory device may be implemented using a semiconductor storage medium such as, for example, ROM and/or RAM, and the auxiliary memory device may be implemented based on a device capable of permanently or semi-permanently storing data, such as a flash memory device (a Solid State Drive (SSD), etc.), a Secure Digital (SD) card, a Hard Disc Drive (HDD), a compact disk, a Digital Versatile Disk (DVD), a laser disk, etc.

Hereinafter, a method for estimating human posture according to an embodiment of the present invention will be described with reference to the drawings.

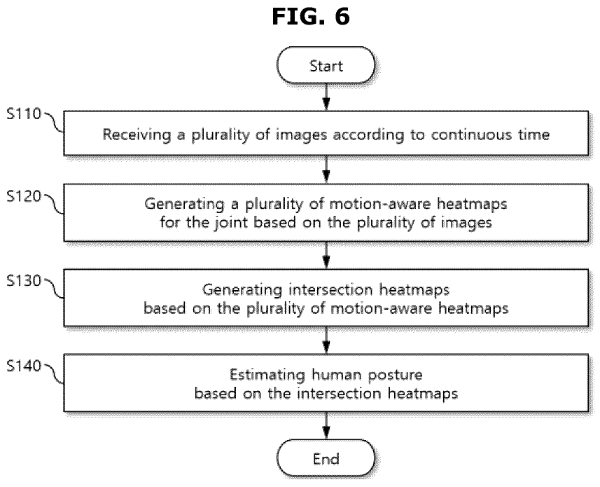

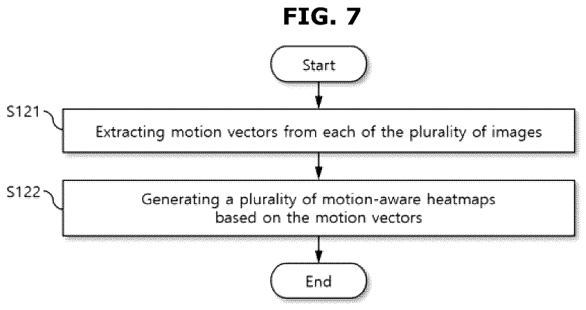

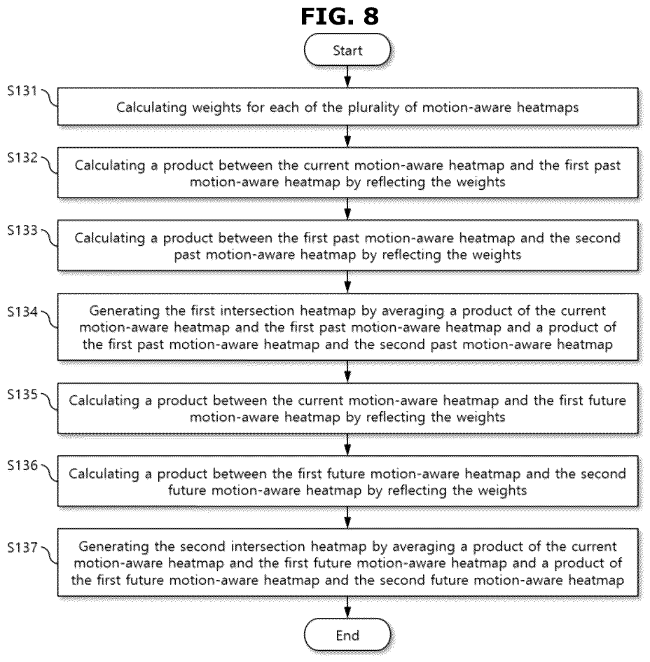

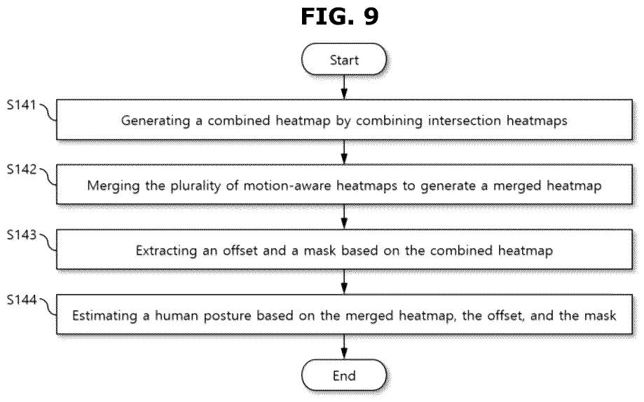

FIG. 6 is a flowchart illustrating a method for estimating human posture according to an embodiment of the present disclosure, FIG. 7 is a flowchart illustrating generation of motion-aware heatmaps according to an embodiment of the present disclosure, FIG. 8 is a flowchart illustrating generation of intersection heatmaps according to an embodiment of the present disclosure, and FIG. 9 is a flowchart illustrating estimation of human posture according to an embodiment of the present disclosure.

As shown in FIG. 6, the inputter 100 receives a plurality of images according to continuous time (S110).

Here, the plurality of images may be continuous frames, and the images and the frames may be used interchangeably.

In an exemplary embodiment of the present invention, it is described that five images are used, but the present invention is not limited thereto.

Referring back to FIG. 6, the generator 210 generates a plurality of motion-aware heatmaps for the joint based on the plurality of images (S120).

As shown in FIG. 7, the generator 210 may extract motion vectors of joint keypoints from each of the plurality of images (S121).

The generator 210 may extract an intermediate feature for each of the plurality of images using a backbone network, and may extract a motion vector based on the extracted intermediate feature.

The generator 210 may generate a plurality of motion-aware heatmaps at different time points based on the respective motion vectors extracted from the plurality of images 111 (S122).

The generator 210 may generate motion-aware heatmaps at a current time point, a past motion-aware heatmap at a past time point based on the current time point, and a future motion-aware heatmap at a future time point based on the current time point.

The generator 210 may generate a plurality of motion-aware heatmaps based on the magnitude and direction of the motion vector of the joint keypoint.

The generator 210 may generate motion-aware heatmaps from each motion vector using a plurality of heads, which are heatmap generators, in order to simultaneously generate the motion-aware heatmaps.

When there are a plurality of motion-aware heatmaps of each of the past time point and the future time point, the generator 210 may generate a motion-aware heatmap H_{c} of a current time point, a first past motion-aware heatmap H_{c→p1} at a first past time point based on the current time point, a second past motion-aware heatmap H_{c→p2} at a second past time point, which is a time point before the first past time point, a first future motion-aware heatmap H_{c→n1} at a first future time point based on the current time point, and a second future motion-aware heatmap H_{c→n2} at a second future time point, which is earlier than the first future time point.

Referring back to FIG. 6, the generator 210 generates intersection heatmaps based on the plurality of motion-aware heatmaps (S130).

The generator 210 may generate the intersection heatmaps in consideration of motions between motion-aware heatmaps of different viewpoints from among the plurality of motion-aware heatmaps.

As shown in FIG. 8, the generator 210 may calculate weights for each of the plurality of motion-aware heatmaps (S131).

The generator 210 may calculate a product between the motion-aware heatmap at the current time and the first past motion-aware heatmap by reflecting the weights for each of the plurality of motion-aware heatmaps (S132).

The generator 210 may calculate a product between the first past motion-aware heatmap and the second past motion-aware heatmap by reflecting the weights for each of the plurality of motion-aware heatmaps (S133).

The generator 210 may generate the first intersection heatmap by averaging a product of the motion-aware heatmap of the current time point and the first past motion-aware heatmap and a product of the first past motion-aware heatmap and the second past motion-aware heatmap (S134).

The generator 210 may calculate a product between the motion-aware heatmap of the current time and the first future motion-aware heatmap by reflecting the weight for each of the plurality of motion-aware heatmaps (S135).

The generator 210 may calculate a product between the first future motion-aware heatmap and the second future motion-aware heatmap by reflecting the weight for each of the plurality of motion-aware heatmaps (136).

The generator 210 may generate the second intersection heatmap by averaging a product of the motion-aware heatmap of the current time point and the first future motion-aware heatmap and a product of the first future motion-aware heatmap and the second future motion-aware heatmap (S137).

That is, the generator 210 may generate the first intersection heatmap based on the second past motion-aware heatmap and the first past motion-aware heatmap, the first past motion-aware heatmap, and the motion-aware heatmap at the current time by reflecting the weight for each of the plurality of motion-aware heatmaps.

In addition, the generator 210 may generate the second intersection heatmap based on the second future motion-aware heatmap and the first future motion-aware heatmap, the first future motion-aware heatmap, and the motion-aware heatmap of the current time by reflecting the weight for each of the plurality of motion-aware heatmaps.

Since steps S120 and S130 have already been described in the generator 210 of FIGS. 1 and 2, a detailed description thereof will be omitted.

Referring back to FIG. 6, the estimator 230 estimates human posture including the location of the human joint based on the intersection heatmaps at step S140.

As shown in FIG. 9, the generator 210 may generate a combined heatmap by combining intersection heatmaps (S141).

The generator 210 may merge the plurality of motion-aware heatmaps to generate a merged heatmap (S142).

The estimator 230 may extract an offset and a mask based on the combined heatmap (S143).

Here, the estimator 230 may extract the offset and the mask through the offset convolution and the mask convolution, respectively, by using the combined heatmap.

The estimator 230 may estimate a human posture based on the merged heatmap, the offset, and the mask (S144).

Here, the estimator 230 may estimate the human posture by inputting the merged heatmap, the offset, and the mask to the deformable convolution.

Operation S140 has already been described in the estimator 230 of FIGS. 1 and 2, and thus a detailed description thereof will be omitted.

As described above, according to the present disclosure, it is possible to robustly estimate a posture even with various changes in human motion, and it is possible to mitigate jittering noise of each joint occurring in a deep learning-based posture estimation model.

Those skilled in the art to which the present invention pertains will understand that the embodiments described herein may be implemented in modified forms without departing from the essential characteristics of the present disclosure. Therefore, the disclosed methods should be regarded from an explanatory perspective rather than a limiting one. The scope of the present invention is defined by the claims rather than the detailed description, and all differences within the equivalent scope of the claims should be construed as being included within the scope of the present invention.

What is claimed is:

1. A method for estimating human posture, the method comprising:
generating a plurality of motion-aware heatmaps for each joint based on a plurality of previously input images corresponding to continuous time;
generating intersection heatmaps by considering motions between motion-aware heatmaps at different time points from among the plurality of motion-aware heatmaps; and
estimating a human posture based on the intersection heatmaps.

2. The method for estimating human posture of claim 1, wherein the generating of the intersection heatmaps comprises:
generating the intersection heatmaps based on motion-aware heatmaps at each of a current time point, a past time point, and a future time point, which are different time points.

3. The method for estimating human posture of claim 2, wherein the generating of the plurality of motion-aware heatmaps comprises:
generating a motion-aware heatmap at the current time point, a past motion-aware heatmap at a past time point relative to the current time point, and a future motion-aware heatmap at a future time point relative to the current time point.

4. The method for estimating human posture of claim 2, wherein the generating of the plurality of motion-aware heatmaps comprises:
when there are a plurality of motion-aware heatmaps of each of the past time point and the future time point, generating current motion-aware heatmaps with respect to the current time point, a first past motion-aware heatmap at a first past time point relative to the current time point, a second past motion-aware heatmap at a second past time point earlier than the first past time point, a first future motion-aware heatmap at a first future time point relative to the current time point, and a second future motion-aware heatmap at a second future time point later than the first future time point.

5. The method for estimating human posture of claim 4, wherein the generating the intersection heatmaps comprises:
generating a first intersection heatmap by averaging a product between the second past motion-aware heatmap and the first past motion-aware heatmap and a product between the first past motion-aware heatmap and the current motion-aware heatmap; and
generating a second intersection heatmap by averaging a product between the second future motion-aware heatmap and the first future motion-aware heatmap and a product between the first future motion-aware heatmap and current the motion-aware heatmap.

6. The method for estimating human posture of claim 1, further comprising:
calculating weights for each of the plurality of motion-aware heatmaps;
wherein the generating the intersection heatmaps comprises:
generating the intersection heatmaps by reflecting the weights of each of the plurality of motion-aware heatmaps.

7. The method for estimating human posture of claim 1, wherein the estimating the human posture comprises:
generating a combined heatmap by combining the intersection heatmaps; and
estimating the human posture on the basis of the combined heatmap.

8. The method for estimating human posture of claim 7, wherein the estimating the human posture on the basis of the combined heatmap comprises:
generating a merged heatmap by merging the plurality of motion-aware heatmaps;
extracting an offset and a mask based on the combined heatmap; and
estimating the human posture based on the merged heatmap, the offset, and the mask.

9. The method for estimating human posture of claim 1, wherein the generating the plurality of motion-aware heatmaps comprises:
extracting a motion vector of a joint keypoint from each of the plurality of images; and
generating the plurality of motion-aware heatmaps based on a magnitude and a direction of the motion vector of the joint keypoint.

10. The method for estimating human posture of claim 1, wherein the generating the plurality of motion-aware heatmaps comprises:
learning to generate the motion-aware heatmaps by using regression loss.

11. An apparatus for estimating human posture, the apparatus comprising a processor,
wherein the processor comprises:
a generator configured to generate a plurality of motion-aware heatmaps for each joint based on a plurality of previously input images corresponding to continuous time, and to generate intersection heatmaps by considering motions between motion-aware heatmaps at different time points from among the plurality of motion-aware heatmaps; and
an estimator configured to estimate human posture in an image at the current time point based on the intersection heatmaps.

12. The apparatus for estimating human posture of claim 11, wherein the generator is further configured to:
generate the intersection heatmaps based on motion-aware heatmaps at each of a current time point, a past time point, and a future time point, which are different time points.

13. The apparatus for estimating human posture of claim 12, wherein the generator is further configured to:
generate a motion-aware heatmap at the current time point, a past motion-aware heatmap at a past time point relative to the current time point, and a future motion-aware heatmap at a future time point relative to the current time point.

14. The apparatus for estimating human posture of claim 12, wherein the generator is further configured to:
when there are a plurality of motion-aware heatmaps of each of the past time point and the future time point, generate current motion-aware heatmaps with respect to the current time point, a first past motion-aware heatmap at a first past time point relative to the current time point, a second past motion-aware heatmap at a second past time point earlier than the first past time point, a first future motion-aware heatmap at a first future time point relative to the current time point, and a second future motion-aware heatmap at a second future time point later than the first future time point.

15. The apparatus for estimating human posture of claim 14, wherein the generator is further configured to:
generate a first intersection heatmap by averaging a product between the second past motion-aware heatmap and the first past motion-aware heatmap and a product between the first past motion-aware heatmap and the current motion-aware heatmap and
generate a second intersection heatmap by averaging a product between the second future motion-aware heatmap and the first future motion-aware heatmap and a product between the first future motion-aware heatmap and current the motion-aware heatmap.

16. The apparatus for estimating human posture of claim 11, wherein the generator is further configured to:
calculate weights for each of the plurality of motion-aware heatmaps, and
generate the intersection heatmaps by reflecting the weights of each of the plurality of motion-aware heatmaps.

17. The apparatus for estimating human posture of claim 11, wherein the estimator is further configured to:
generate a combined heatmap by combining the intersection heatmaps and
estimate the human posture on the basis of the combined heatmap.

18. The apparatus for estimating human posture of claim 17, wherein the estimator is further configured to:
generate a merged heatmap by merging the plurality of motion-aware heatmaps,
extract an offset and a mask based on the combined heatmap and
estimate the human posture based on the merged heatmap, the offset, and the mask.

19. The apparatus for estimating human posture of claim 11, wherein the generator is further configured to:
extract a motion vector of a joint keypoint from each of the plurality of images and
generate the plurality of motion-aware heatmaps based on a magnitude and a direction of the motion vector of the joint keypoint.

20. The apparatus for estimating human posture of claim 11, wherein the processor further comprises:
a learner configured to learn to generate the motion-aware heatmaps using regression loss.
